(19) United States
(12) Patent Application Publication
(10) Pub. No.: US 2001/0041716 A1
(43) Pub. Date: Nov. 15, 2001

(54) Compositions and methods for locally treating inflammatory diseases

(76) Inventors: Timothy J. Laing, Ann Arbor, MI (US); Gordon L. Flynn, Ann Arbor, MI (US)

Correspondence Address: LAHIVE & COCKFIELD, 28 STATE STREET, BOSTON, MA, 02109, US

(21) Appl. No.: 09/728,884

(22) Filed: Dec. 1, 2000

Related U.S. Application Data
(60) Provisional application No. 60/168,561, filed on Dec. 2, 1999.

(51) Int. Cl. A61K 31/47; A01N 43/42

(52) U.S. Cl. 514/310

(57) ABSTRACT

Compositions and methods are provided for treating inflammatory diseases in mammals by inhibiting TNFα expression. The methods comprise the step of topically administrating a composition of the present invention comprising thalidomide, N-alkyl analogs of thalidomide and combinations thereof.

### FIELD OF THE INVENTION

The present invention relates generally to compositions and methods for locally treating inflammatory diseases and, more particularly, to compositions and methods for treating inflammatory diseases by percutaneous delivery of thalidomide and analogs thereof.

### BACKGROUND OF THE INVENTION

Thalidomide is a piperidinedione immunomodulator and is derived from a natural endogenous α-amino acid (glutamic acid). It is described as a N-phthaloyl-glutamic acid imide, and the chemical name is a-phthalimidoglutarimide. It was developed in the 1950's as the first example of a new class of non-barbiturate sedatives. Unfortunately, it was found to cause birth defects in infants born to women who had taken thalidomide during pregnancy. It was withdrawn from the market in 1961, but still remained available in certain countries for research purposes. Subsequently, and despite its history, thalidomide has been found to be beneficial in treating more than 20 different diseases including leprosy, tuberculosis, AIDS and various autoimmune diseases (Stirling, D. et al., Journal Of The American Pharmaceutical Association NS37:307-313 (1997)). In addition, thalidomide is currently used world-wide for the treatment of Bechet's disease and is the single most effective agent against erythema nodosum leprosum (ENL), a severe inflammatory skin condition associated with leprosy, (Sheskin, J., Clinical Pharmacology And Therapeutics 6:303-306 (1965)).

It was only recently that thalidomide's probable mechanism of action in ENL was uncovered and the therapeutic potential of the drug in other chronic inflammatory disorders appreciated. It appears that thalidomide's therapeutic effects in ENL are due to it's ability to reduce levels of tumor necrosis factor-alpha (TNFα). (Sampaio, E. P. et al., Journal Of Experimental Medicine 173:699-703 (1991)). Thalidomide significantly down-regulates the production of TNFα, mainly by affecting peripheral blood mononuclear cells (PBMC's), when stimulated with an appropriate agonist, e.g., microbial lipopolysaccharide (LPS). It appears to accomplish this without affecting the production of other known essential cytokines.

TNFα is one of a number of cytokines that is essential to immunological responses in which inflammation is observed. It is produced by a variety of cell types, most notably mononuclear cells (macrophages and monocytes), immune system cells which are principal effectors of inflammation. Though inflammation is the normal immune system response to infection or injury, serving to rid the body of foreign agents and to clear wounds of dead and dying tissue, chronic over-expression of TNFα leads to either a persistent or an overly robust inflammatory response. For example, it is elevated levels of this cytokine which are known to be responsible for the wasting of tissue that occurs in ENL. It is therefore not surprising that it has been shown to be useful in treating other diseases, such as AIDS, where tissue wasting is part of the disease expression.

Consequently, TNFα's over-production has been tied directly to the debilitating symptoms of infectious diseases and to certain immune-related disorders such as rheumatoid arthritis (RA), where the wasting of tissue at joints is crippling. In the case of RA, TNFα has been detected in synovial tissue and fluid of afflicted joints. The generation of TNFα locally within the RA joint has also been confirmed histologically using in situ hybridization techniques and immunostaining. These studies have indicated that cells of the monocyte/macrophage lineage appear to be the principal source of TNFα within the synovium, although other cells, e.g., T-cells and endothelial cells, also contribute. (Chu, C. Q. et al., Arthritis and Rheumatism 34:1125-1132 (1991)). The initial relief of systemic symptoms brought about upon administration of thalidomide in RA is coupled with a concomitant drop in TNFα levels in rheumatoid arthritis patients (Stirling, D. I., Pharmaceutical News 3:307-313 (1996)). The identification of TNFα as a key mediator of inflammation in RA has led to randomized clinical trials using anti-TNFα monoclonal antibody as a drug, producing beneficial results (Elliott, M. J. et al., The Lancet 344:1105-1110 (1994);, Rankin, E. C. et al., British Journal Of Rheumatology 34:334-342 (1995)). In addition, a chimeric humanized antibody (ETANERCEPT) is able to neutralize circulating TFNα by simulating soluble TNFα receptors. ETANERCEPT has been approved by the FDA and is commonly used for treatment of RA. Taken together, these observations support the hypothesis that reducing TNFα concentrations is an attractive goal in the treatment of RA.

The etiology of RA remains unclear, but it is known that the synovial membranes lining the joints are infiltrated by large numbers of immunologically active cells, including lymphocytes. During this process, multiple inflammatory cytokines are elaborated into the synovial fluid, which exert destructive effects on articular cartilage and ultimately compromise joint function. Because of the mass of infiltrated inflammatory cells, the joint swells and feels distended and pliant to the touch. Increased blood flow, a feature of the inflammation, makes the joint warm. Any joint can be affected, but the wrists and knuckles are almost always involved and often the knees and the joints of the ball of the foot. In the absence of proper treatment, crippling joint deformities can result. Patients with RA describe feeling much like they have a virus, with fatigue and aching in the muscles, except that, unlike a usual viral illness, the condition tends to persist for months or even years (Fries, J. F., Arthritis: A Comprehensive Guide To Understanding Your Arthritis USA:Addison-Wesley Publishing Co. (1986)). Although it can begin at any age, the condition usually appears in midlife. Since RA is common and sometimes severe, it is a major global health problem, affecting about one percent of the population worldwide. RA accounts for more disability expenditures by the U.S. federal government than any other disease.

Tissue-wasting and other damage caused by arthritis can be reduced or even stopped with oral use of thalidomide. However, the high systemic levels of orally-administered thalidomide that are required to alleviate RA-related symptoms often cause a number of unwanted side effects. Drowsiness, constipation, eosinophilia, swelling of the lower limbs and, significantly, peripheral neuropathy are easily provoked. (Gutierrez-Rodriguez, O., Arthritis and Rheumatism 27:1118-1121 (1984)).

To avoid the unwanted side effects of systemic delivery, it would thus be highly advantageous to provide a method of locally delivering thalidomide and analogs thereof to treat inflammatory diseases such as RA. It would also be desirable to provide thalidomide analogs with improved physiochemical properties for local delivery.

### SUMMARY OF THE INVENTION

Compounds, compositions and methods for treating inflammatory conditions associated with increased expression of TNFα in mammalian tissue are provided. The methods comprise percutaneous delivery of thalidomide and thalidomide analogs to inhibit inflammation. Thalidomide and thalidomide analogs inhibit inflammation by diffusing into tissues and inhibiting the production of TNFα.

Administration of thalidomide and thalidomide analogs via the dermal route bypasses liver metabolism and provide effective local tissue drug levels without systemic complications. Thalidomide's action is thought to be on TNFα's expression at the local tissue level. Therefore, by local delivery, localized inflammation may be selectively treated. The percutaneous, localized delivery methods of the present invention are particularly effective in the treatment of rheumatoid arthritis where the joints in extremities are most effected, as well as in the cutaneous manifestations of systemic lupus erythematosus (SLE). Thus, local applications of the thalidomide and thalidomide analog compositions described herein, down-regulate TNFα production in and around the delivery site without incurring systemic levels of such agents.

Additional objects, advantages, and features of the present invention will become apparent from the following description and appended claims, taken in conjunction with the accompanying drawings.

### BRIEF DESCRIPTION OF THE DRAWINGS

The various advantages of the present invention will become apparent to one skilled in the art by reading the following specification and subjoined claims and by referencing the following drawings in which:

FIG. 1 is a graph showing the relationship between the melting points of thalidomide and three N-alkyl analogs and alkyl chain length;

FIG. 2 is a graph showing the log partition coefficients of thalidomide and three N-alkyl analogs as a function of alkyl chain length;

FIG. 3 is a graph showing the relationship between aqueous solubility of thalidomide and three N-alkyl analogs and alkyl chain length;

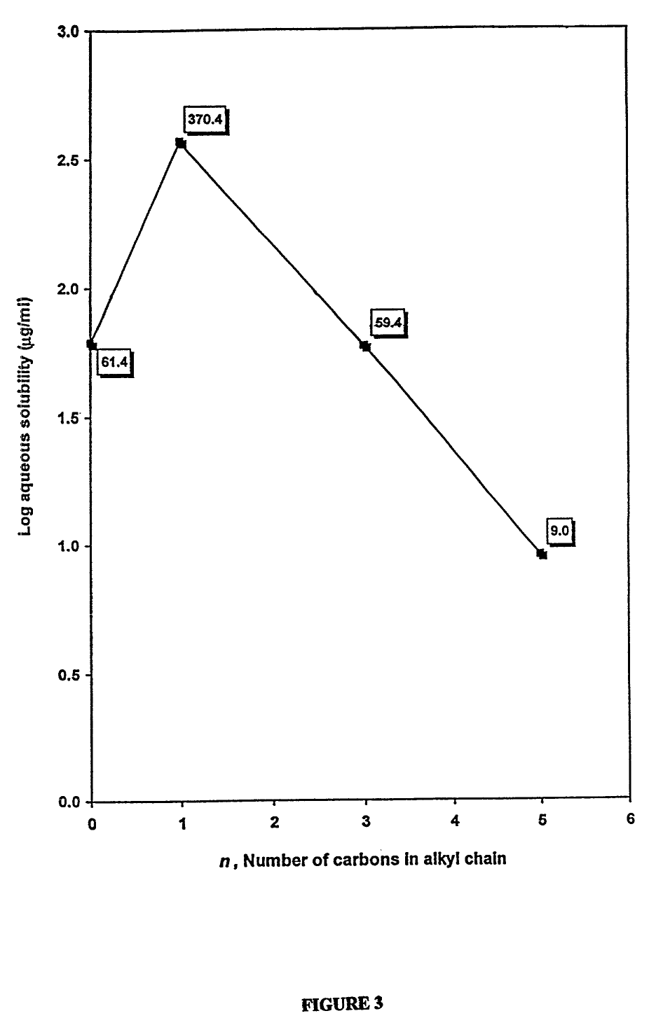

FIG. 4 is a graph showing the relationship between permeability coefficient of thalidomide and three N-alkyl analogs and partition coefficient;

FIG. 5 is a graph showing the steady-state fluxes of thalidomide and its N-alkyl analogs through human cadaver skin from water at 32° C.;

FIG. 6 is a graph showing the representative permeation profiles of thalidomide and its N-alkyl analogs through human cadaver skin from formulation C (see Table 6) at 32° C.;

FIG. 7 is a graph showing the steady-state fluxes of thalidomide and its N-alkyl analogs through human cadaver skin from saturated n-alcohol solutions as a function of alcohol chain length;

FIG. 8 is a graph showing the solubilities of thalidomide and its N-alkyl analogs in various formulations (see Table 6);

FIG. 9 is a graph showing the steady state flux of thalidomide and its N-alkyl analogs through human cadaver skin from various formulations (see Table 6);

FIG. 10 is a graph showing the permeation profiles of N-methylthalidomide through human cadaver skin from various formulations (see Table 6);

FIG. 11 is a graph showing the inhibition of TNFα by thalidomide and its N-alkyl analogs.

### DETAILED DESCRIPTION OF THE PREFERRED EMBODIMENTS

Compositions and methods for treating inflammatory diseases associated with the over-production of TNFα are provided. In one embodiment, a method of the present invention comprises treating a mammal, and especially a human patient, diagnosed with an inflammatory disease by percutaneously delivering an effective amount of thalidomide. In another embodiment, a method of the present invention comprises treating a mammal diagnosed with an inflammatory disease by percutaneously delivering an effective amount of an analog of thalidomide. In yet another embodiment, a method of treating a mammal diagnosed with an inflammatory disease is provided, wherein an effective amount of an N-alkyl analog of thalidomide is percutaneously delivered to the mammal. The N-alkyl thalidomide analogs are preferably C₁-C₁₀, wherein C₁-C₅ is preferred and C₁-C₃ is most preferred. It will be appreciated that mixtures of the compounds of the present invention may also be employed.

The compounds of the present invention have physiochemical properties advantageous to percutaneous delivery. It is well established that compounds favored for percutaneous delivery have low molecular weights, a low level of crystallinity as reflected by a low melting temperature and reasonable solubilities in both hydrophilic and hydrophobic solutions. In a preferred embodiment, the compounds have a K octane/water between about 10 and about 10,000, preferably between about 10 and about 5,000, more preferably between about 10 and about 1,500. Likewise, preferred compounds have a melting point below about 200° C., preferably below about 160° C., and more preferably below about 100° C. In a preferred embodiment, the thalidomide analog is fluorinated.

While not wishing to be bound by theory, it is believed that the addition of N-alkyl groups to thalidomide, as described herein, lowers the level of the crystallinity of thalidomide, decreases the melting temperature, and increases lipophilicity, thereby increasing the abilities of the analogs to diffuse across membranes. Those skilled in the art, with the teachings of the present invention, will recognize that a combination of potency and delivery capabilities are requisite and will be able to identify compounds embodied by the present invention appropriately balancing these attributes.

Pharmaceutical compositions comprising the compounds of the present invention are also provided. In one embodiment, the composition comprises a percutaneous penetration enhancer that augments delivery of the thalidomide and the analogs thereof also referred to herein as the “active ingredient,” to the subcutaneous layers where the inflammation is found. Percutaneous penetration enhancers include both chemical and physical enhancers, and mixtures thereof. Chemical penetration enhancers are added in amounts effective to deliver active ingredients to subcutaneous layers.

Any chemical penetration enhancer known to the skilled artisan may be employed in the compositions of the present invention. Preferred enhancers are those that are pharmacologically and chemically inert and chemically stable, potent, nonirritating, nonsensitizing, nontoxic, nonallergenic, odorless, tasteless, colorless and cosmetically acceptable. Chemical penetration enhancers include, but are not limited to, sulfoxides such as dimethylsulfoxide, glycols such as propyleneglycol, fatty acids and esters such as 1,3 butylene glycol-1-monolaurate, short chain and/or unsaturated fatty acids such as , N-methylpyrrolidone and simple solvents such as water and alcohols.

Physical penetration enhancers may also be employed either alone, or in combination, with chemical percutaneous penetration enhancers. Physical enhancers include, but are not limited to, those enhancing skin hydration, occlusion devices, hydrocolloid patches and other transdermal delivery polymers. Also included are methods involving the use of liposomes, delipidization techniques, electroporation, ultrasound, iontophoresis and like methods known to those skilled in the art which increase skin permeability without toxicity and destruction of the stratum corneum.

The pharmaceutical compositions of the present invention may further comprise a pharmaceutically acceptable carrier for topical application. The term “pharmaceutically acceptable” means an essentially non-toxic material that does not interfere with the effectiveness of the biological activity of the active ingredients, but rather assures such quality or qualities.

As used herein the term “topical administration” means directly applying or spreading a formulation on epidermal tissue, especially the outer skin. The terms “therapeutically effective amount” and “therapeutically effective duration” mean the total amount of each active component of the pharmaceutical composition and a duration of treatment that are sufficient to show a meaningful patient benefit, i.e., treatment, healing, prevention or amelioration of the relevant medical condition, or an increase in the rate of treatment, healing, prevention or amelioration of such condition without undue adverse physiological effects or side effects. The term “therapeutically effective amount” when applied to an individual active ingredient administered alone refers to that ingredient alone. When applied to a combination, the term refers to combined amounts of the active ingredients, e.g., thalidomide and analogs, that result in the therapeutic effect, whether administered in combination, serially or simultaneously.

In practicing the methods of the present invention, a therapeutically effective amount of a thalidomide, thalidomide analog or a combination thereof, is administered to a patient having a condition to be treated, i.e. a condition associated with chronic or high levels of TNF α production. Examples of inflammatory disease associated with chronic or high levels of TNFα production that can be treated with the methods of the present invention include, without limitation, inflammatory arthritis such as rheumatoid, psoriatic, reactive, viral or post-viral arthritis and skin conditions such as psoriasis, erythema nodosum leprosum (ENL), cutaneous manifestations of SLE, and Bechet's disease. Thalidomide and the thalidomide analogs of the present invention may be administered in accordance with the methods of the invention either alone or in combination and also in combination with other conventional therapies.

When thalidomide and thalidomide analogs are administered topically by the methods of the present invention, they are typically applied in an admixture with a dermatologically acceptable carrier or vehicle (e.g., as a lotion, cream, ointment, soap, or the like). This may contain a percutaneous penetration enhancer or mixture of enhancers. When a carrier is employed, it is necessary that the carrier be inert in the sense of not bringing about a deactivation of active ingredients, and in the sense of not bringing about any adverse effect on the skin to which it is applied. Many preferred carriers finction well as penetration enhancers to augment the effect of other ingredients so that active ingredients are efficiently delivered to subcutaneous tissue.

Suitable carriers include water, alcohols, oils, lipids and the like, chosen for their ability to dissolve or disperse the active ingredients at concentrations of active ingredients most suitable for use in the therapeutic treatment. Generally, even low concentrations of active ingredients in a carrier will be suitable, requiring only that more frequent topical application be resorted to. As a practical manner, however, to avoid the need for repeated application, it is desirable that the topically applied composition (e.g., thalidomide and/or analogs in association with other ingredients in a carrier) be formulated to contain at least about 0.001% to about 5.0% by weight, thalidomide and or analogs thereof, preferably about 0.01% to about 2.0% and more preferably about 0.1% to about 1.0%. Carriers are chosen which solubilize or disperse active ingredients at such concentrations, and in some cases to penetrate the skin to deliver them to subcutaneous muscle tissue. Preferably, the carrier will comprise an optimized admixture of water, ethanol and n-octanol, more preferably between about 0.0% and about 75% of water, between about 0.0%, and about 25.0%, preferably 25.0%, about 0% to about 100% ethanol, and between about 0.0% and about 5.0% n-octanol.

While the carrier for thalidomide and/or thalidomide analogs may consist of a relatively simple solvent or dispersant such as a hydroalcaholic vehicle or a simple oil, it is generally preferred that the carrier be one which aids in percutaneous delivery and penetration of the active ingredients into lipid layers and subcutaneous muscle tissue as discussed above. Moreover, the composition preferably is one that is conducive to topical application, and particularly one that can be applied so as to localize the application. Many such compositions are known in the art, and can take the form of lotions, creams, ointments, gels or even solid compositions (e.g., stick-form preparations). Typical compositions include lotions containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phopholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophillic colloids. Such compositions are referred to herein as “dermatologically acceptable carriers.” Most preferred carriers for use with active ingredients of the present invention are fat-soluble, i.e., those which can effectively penetrate skin layers and deliver the active ingredients to the lipid-rich layers of the skin and to subcutaneous muscle tissue.

It will be appreciated that the amount of thalidomide and/or thalidomide analog in the pharmaceutical composition of the present invention will vary depending upon the nature and severity of the condition being treated, and on the nature of prior and/or concurrent treatments which the patient has undergone or is undergoing, as well as the potency of the compound employed and its ability to penetrate the skin. Ultimately the attending physician will determine the amount of the pharmaceutical composition of the present invention with which to treat each individual patient based on the above mentioned patient-specific criteria as well as the character of the compound (thalidomide and/or analogs thereof). It is understood that the character of the compound is a function both of its intrinsic pharmaceutical potency and of its ability to penetrate the skin. Initially the attending physician will administer low doses of thalidomide and/or thalidomide analog and observe the patient's response. Larger doses of thalidomide and/or thalidomide analog may be administered until the optimal therapeutic effect is obtained for the patient, and at that point the dosage is not increased further.

The foregoing and other aspects of the invention may be better understood in connection with the following examples, which are presented for purposes of illustration and not by way of limitation.

### SPECIFIC EXAMPLE 1

Physiochemical Properties and Solubility Analysis of Thalidomide and N-alkyl Analogs

I. Background

It is important to have a thorough understanding of a drug's physicochemical properties, particularly its absolute and relative solubilities and related partitioning tendencies, in order to evaluate the drug for percutaneous delivery. (Sloan, K. B. et al., Journal Of Investigative Dermatology 87:244-252 (1986); Flynn, G. L. et al., Journal Of Pharmaceutical Sciences 61:838-852 (1972)). Since membrane permeation is a function of skin/permeant and solvent/permeant interactions, an effort has been made to model absorption through skin by quantitating these interactions using partition coefficients and solubility parameters, as well as parameters describing crystallinity and other physical properties which are predictive of diff-usion rates and gradients. A drug's behavior relative to its dose may dictate the type of physical system most appropriate for administration of the drug. Two reference behaviors were employed in the solubility analysis of the compounds of the present invention, primarily, ideal solution behavior and secondarily, regular solution behavior. An ideal drug being delivered percutaneously should have a low molecular weight (e.g <500 g/mole). A high level of crystallinity is expressed in the form of a high melting point and high heat of fusion. This limits solubility itself, and thus also sets a limit on mass transfer across the skin. Generally, the greater the innate tendency of a drug to dissolve, the more likely it is that the drug can be delivered at an appropriate rate across the skin. Therefore, with all other factors being equal, a low melting point is preferred.

Absolute solubilities and partition coefficients (relative solubilities) are the major determinants of a drug's dissolution and distribution between phases with which it contacts, and, thus, its bioavailability. The hydrophobicity of a compound is a key determinant of its ease of skin transport. Hydrophobicity is well reflected in the relative abilities of drugs to partition between “oil” and water. The stratum corneum has for many years been identified as, to a first good approximation, a nonpolar membrane. Its “solvent” properties have therefore been mimicked by various nonpolar liquids including hexane, ether and octanol. A drug having been released from a topical formulation, will partition into the stratum corneum, diffuse across this tissue and then partition into the underlying epidermis. Alternatively, a drug may bypass the stratum corneum by partitioning into and diffusing through the skin's appendages. However, when a drug reaches the viable tissue, it encounters a phase change; it has to transfer from the predominantly lipophilic intercellular channels of the stratum corneum or the sebum filling the hair follicle into the living cells of the epidermis, which will be largely aqueous in nature. Therefore, skin permeants must have reasonable solubilities in oil and water, but should favor oil. A preferentially oil soluble drug may have difficulty leaving the stratum corneum or sebum, while on the other hand an extremely polar drug will have trouble partitioning into the stratum corneum from its vehicle.

The lipophilicities of thalidomide and its N-alkyl analogs and their solubilities in select solvents were assessed along with other important solubility-determining properties such as melting points, fusion energies and molecular cohesiveness. These properties are all determinative of the ease of delivery of the compounds through skin.

II. Synthesis

Thalidomide, N-methyl thalidomide, N-propyl thalidomide and N-pentyl thalidomide were synthesized according to literature methods (Budavari, S., ed. The Merck Index, 11^{th} Ed. Rahway, N. J.: Merck (1989) and De, A. U. et al., Journal Of Pharmaceutical Sciences 64:262-266 (1975)). Identification and levels of purity (>96%) were assured through Element Analysis (EA), Electron Impact Mass Spectroscopy (MS), Nuclear Magnetic Resonance (NMR) spectroscopy, High-pressure Liquid Chromatography (HPLC) and by the sharpness of melting points.

39 grams of N-phthaloyl-DL-glutamic anhydride (Aldrich, Milwaukee, Wis., USA) were fused with 18 grams of urea (Aldrich, Milwaukee, Wis., USA) in an oil bath at 170-180° C. for 45 min. The crude thalidomide was recrystallized from ethanol. 20 grams of N-phthaloyl-DL-glutamic anhydride (and 46.32 ml of a 2 M methylamine solution in methyl alcohol (Sigma Chemical Co., St. Louis, Mont., USA) were heated in an oil bath at 200° C. for 8 hr. After cooling, the crude imide was purified by recrystallization from 95% ethanol (De, A. U. et al., Journal Of Pharmaceutical Sciences 64:262-266 (1975)). According to HPLC, elemental analysis and the sharpness of the melting point (133° C.), the N-methyl thalidomide was >98% pure.

20 grams of N-phthaloyl-DL-glutamic anhydride and 7.62 ml of propylamine (Sigma Chemical Co., St. Louis, Mont., USA) were heated in an oil bath at 200° C. for 8 hours. The crude imide was purified by recrystallization from EtOH (95%). The melting point of the purified material was 136° C.

20 grams of N-phthaloyl-DL-glutamic anhydride were heated with 10.74 ml of N-amylamine (Sigma Chemical Co., St. Louis, Mont., USA) in an oil bath at 200° C. for 8 hours. The crude N-pentyl thalidomide was purified by silica gel column chromatography (diethyl ether:hexane) using a stepwise gradient (10:90; 20:80; 30:70; 40:60; and 45:55) instead. A melting point of 105° C. was determined for the compound. It was dried in vacuo and recrystallized from diethyl ether.

Quantitative Analytical Procedure: An HPLC assay was developed for quantitative analysis of the compounds. Under the chromatographic conditions employed, the retention times of thalidomide and its N-methyl, N-propyl and N-pentyl analogs were approximately 7, 6, 7, and 8 minutes, respectively. All evidenced single peaks at 220 nm. Calibration curves showed excellent linearity over the entire concentration range.

Solubility Determination: The solubility of thalidomide and its N-alkyl analogs in several organic solvents (hexane, cyclohexane, carbon tetrachloride, toluene and benzene) (Fisher Scientific, Pittsburg, Pa., USA) were obtained by equilibrating large excesses of the solute with each solvent. Temperature was maintained at 25° C. Samples of the slurries were taken periodically and filtered. After careful notation of its volume, each sample was then evaporated to dryness. The solute was redissolved in methanol, appropriately diluted and assayed. No impurities were detected by the HPLC assay.

Differential Thermal Analysis: The heat of fusion (ΔH_{f}) and the entropy of fusion (ΔS_{f}) of thalidomide and its N-alkyl analogs were determined with a Perkin-Elmer DSC7 Differential Scanning Calorimeter (DSC), which was calibrated with an indium standard.

Melting Point: The melting points of thalidomide and its N-alkyl analogs were determined by: (1) differential thermal analysis and (2) controlled-heating thermal microscopy.

Determination of Partition Coefficient: The distributions of thalidomide and its analogs were measured between equal volumes of n-octanol and phosphate buffer (pH 6.4) co-saturated with each other. The pH of the buffer was measured before and after each drug was added. The presence of the compounds had no influence on the pH. Partition coefficients (K_{oct}) were calculated as the ratio of drug concentration in the n-octanol phase to that in the buffer phase.

III Results

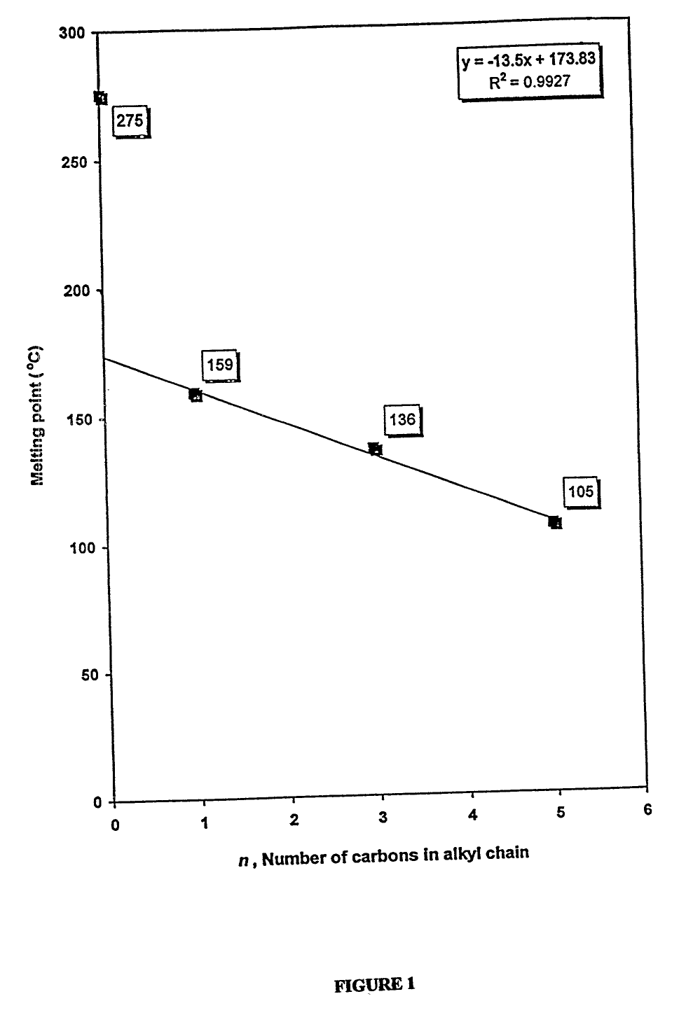

Table 1 summarizes the physicochemical properties of thalidomide and three N-alkyl analogs. The molecular weight of each compound was determined experimentally with electron impact mass spectroscopy (EI-MS). There were no differences between the expected and experimental molecular weights of the compounds. Enthalpies of fusion, ΔH_{f}, and entropies of fusion, ΔS_{f}, calculated from thermoanalytical data are also shown in Table 1. Thalidomide, N-propyl thalidomide and N-pentyl thalidomide each exhibited only one thermal transition. The endotherms at 275, 136 and 105° C., correspond to the melting of these crystals, respectively. N-methyl thalidomide showed an endotherm at 159° C. and a second small endotherm at 165° C. The endotherm at 159° C. corresponds to the melting point of N-methyl thalidomide. Melted samples of all the compounds, assayed by HPLC, showed only trace impurities. FIG. 1 represents the trend in melting points as a function of alkyl chain length.

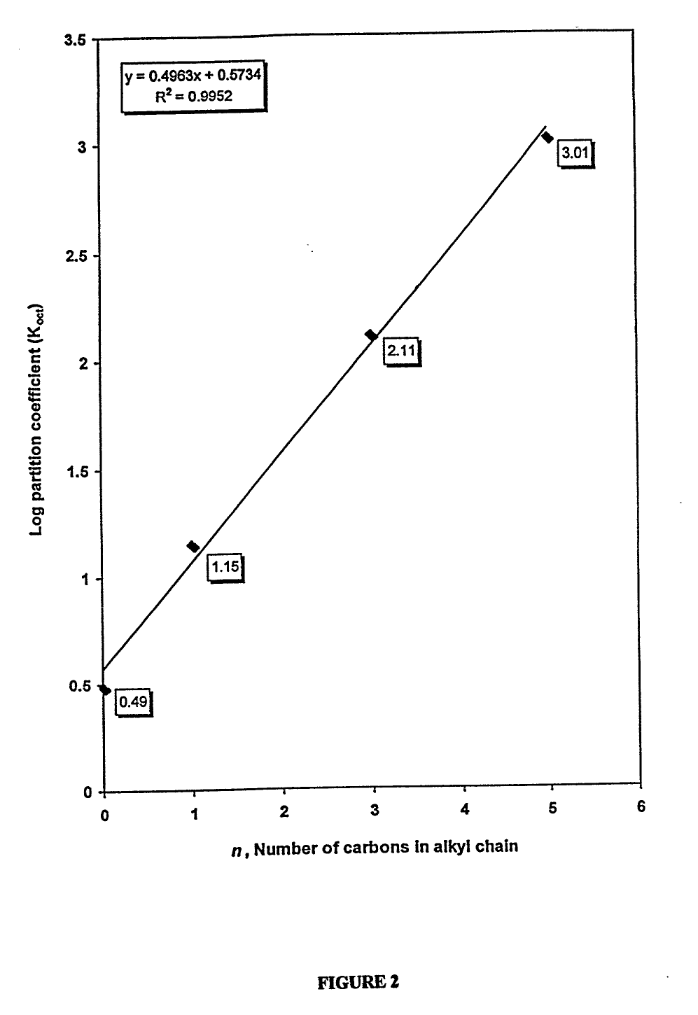

The aqueous solubilities±standard deviation (SD) of thalidomide and its N-alkyl analogs are listed in Table 2, along with their octanol/water (K_{oct}) partition coefficients±standard deviations (SD). N-alkylation of the glutarimide ring in the thalidomide molecule results in compounds (N-methyl, N-propyl and N-pentyl analogs) that are more lipophilic. FIG. 2 illustrates that the log [partition coefficients] (log K_{oct}) increases linearly with increasing alkyl chain length.

TABLE 1
Physicochemical properties of thalidomide and its N-alkyl analogs.

| Physical | Thali- | N-Methyl | N-Propyl | N-Pentyl |
|---|---|---|---|---|
| Parameter | domide | Thalidomide | Thalidomide | Thalidomide |
| Molecular weight | 258 | 272 | 300 | 328 |
| (g/mole) | | | | |
| Crystalline | 1.48 | 1.43 | 1.35 | 1.28 |
| density | | | | |
| (g/ml) | | | | |
| Molar volume, V₂ | 174 | 191 | 223 | 255 |
| (ml/mole) | | | | |
| Melting | 275 ± | 159 ± 0.11 | 136 ± 0.90 | 105 ± 0.26 |
| Temperature, | 0.11 | | | |
| T_{f}(° C.) ± SD | | | | |
| Heat of fusion, | 8.61 ± | 4.33 ± 0.06 | 6.52 ± 0.25 | 5.73 ± 0.08 |
| ΔH_{f} | | | | |
| (kcal/mole) ± SD | 0.27 | | | |
| Entropy of fusion, | 15.71 | 10.02 | 15.94 | 15.16 |
| ΔS_{f} | | | | |
| (cal/mole/K) | | | | |
| Activity of solid | 1.29 × | 1.03 × 10⁻¹ | 5.18 × 10⁻² | 1.29 × 10⁻¹ |
| phase | 10⁻³ | | | |
| a₂^{s}, ΔCₚ = 0* | | | | |
| Activity of solid | 8.07 × | 1.54 × 10⁻¹ | 7.89 × 10⁻² | 1.64 × 10⁻¹ |
| phase | 10⁻³ | | | |
| a₂^{s}, ΔCₚ = ΔSₚ* | | | | |

Ideal activity of solid phase, a₂^{s} estimated from:

$ln {a}_{2}^{s} - \frac{- Δ {H}_{f}}{RT} ( \frac{{T}_{f} - T}{{T}_{f}} ) + \frac{Δ {C}_{p}}{{T}_{f}} ( \frac{{T}_{f} - T}{T} ) - \frac{Δ {C}_{p}}{R} ( ln \frac{{T}_{f}}{T} )$ with one or the other assumption.

TABLE 2
Solubility and partition coefficients of thalidomide and its N-alkyl
analogs.

| | SOLUBILITY (25° C.) | | |
|---|---|---|---|
| | Water (pH 6.4) | Hexane | |
| Compound | μg/ml ± SD | μg/ml ± SD | K_{oct} ± SD |
| Thalidomide | 52.1 ± 1.49 | 0.1 ± 0 | 3.09 ± 1.03 |
| N-Methyl Thalidomide | 275.9 ± 6.39 | 90 ± 0 | 14.1 ± 1.05 |
| N-Pentyl Thalidomide | 57.3 ± 1.46 | 220 ± 10 | 129 ± 1.05 |
| N-Propyl Thalidomide | 6.54 ± 0.52 | 530 ± 10 | 1023 ± 1.06 |

It can be seen in Table 2 that the hexane solubilities of the compounds are low and therefore, the assumption that the volume fraction of hexane in the saturated solutions (φ₁) is unity was made. Using this surmise, the solubility parameters for all the solutes were calculated. The differences in solubility parameters and in the ideal solubility created by the alternate assumptions for ΔCₚ are given in Table 3. To show the extent to which thalidomide's, N-methyl thalidomide's, N-propyl thalidomide's and N-pentyl thalidomide's solubility behavior might conform to regular solution behavior, the regular solution solubility parabolas for all four compounds were calculated about the midpoints of 13.7, 12.3, 11.5 and 11.2 (cal/cm³)^{½}, respectively. In each case the solutions are considered ideal at the peak of their parabolas.

TABLE 3
Comparison of solubility parameters and ideal solubility from
experimental results.

| | | δ₂ (Hexane) | | | |
|---|---|---|---|---|---|
| | Melting Point | (cal/cm³)^{½} | | Ln X_{2,ideal} | |
| Compound | (° C.) | ΔC_{p = 0} | ΔCₚ = ΔSᵣ | ΔCₚ = 0 | ΔCₚ = ΔSᵣ |
| Thalidomide | 275 | 13.2 | 13.7 | −6.65 | −4.82 |
| N-Methyl | 159 | 12.2 | 12.3 | −2.27 | −1.87 |
| N-Propyl | 136 | 11.4 | 11.5 | −2.96 | −2.54 |
| N-Pentyl | 105 | 11.2 | 11.2 | −2.05 | −1.81 |

The N-alkyl analogs melt at lower temperatures than does thalidomide and, in so doing, consume less energy per mole. By adding a methyl group to the thalidomide structure, the melting point drops by over 100° C. and, in this particular instance upon increasing the alkyl chain length to five —CH₂— units, the melting points decrease more or less linearly (FIG. 1). Other investigators who have studied the influence of extending alkyl chain length also report that melting points decrease overall, but often not linearly. (Stinchcomb, A. L. et al., Pharmaceutical Research 12:1526-1529 (1995); Yalkowsky, et al., 1972)). The melting points for the N-alkyl analogs in this series are all at least 100° C. lower than thalidomide's melting point, illustrating the remarkable impact upon crystallization properties through elimination of the acidic imido hydrogen atom of the thalidomide molecule.

N-alkylation of the glutaranide ring in the thalidomide molecule results in compounds (N-methyl, N-propyl and N-pentyl analogs) that are more lipophilic. This is evident from the systematically declining solubility parameters through the series but is even better demonstrated in the octanol/water partition coefficients (Table 2). FIG. 2 illustrates that the log [partition coefficients](log K_{oct}) increases linearly with increasing alkyl chain length. Here one observes a linear free energy relationship which mostly has developed around the incremental excess free energy expected to dissolve —CH₂— groups in water. Similar relationships exist between the water/octanol partition coefficients and solute physical properties of selected narcotic analgesics (Roy, S. D. et al., Pharmaceutical Research 5:580-586 (1988)).

Table 1 contains estimates of relative thermodynamic activities of thalidomide and its N-alkyl analogs at 25° C. These values also represent the respective mole fractional ideal solubilities of thalidomide and its N-alkyl analogs. It can be seen that the inherent thermodynamic activity increases dramatically when the thalidomide structure is alkylated. However, there is no simple pattern to the thermodynamic activities of the analogs as a result of extending the alkyl chain. While it is inappropriate to directly relate the thermodynamic activity of one compound to that of another, as there is no provision in classical thermodynamics for doing so, it is still clear from these data that a high level of crystallinity is associated with low activity and vice versa.

At 52 μg/ml (Table 2), the 25° C. aqueous solubility of thalidomide is exceptionally low. Its low solubility in water is undoubtedly due to its exceptionally high level of crystallinity as reflected in its high melting point and enthalpy of fusion. By way of contrast, the aqueous solubility of N-methyl thalidomide, 276 μg/ml, is quite high. The loss of the H-bonding imido hydrogen is more than compensated for by the reduced crystallinity of the compound. The regular solution solubility parabolas for all four compounds were calculated about the midpoints of 13.7, 12.3, 11.5 and 11.2 (cal/cm³)^{½}, respectively, where in each case the solutions are considered ideal.

In conclusion, alkylation of the thalidomide molecule results in compounds with physicochemical properties that are well suited for percutaneous delivery. The N-alkyl analogs of thalidomide have lower melting points and consume less energy per mole in doing so. They are more lipophilic as evident in their higher octanol/water partition coefficients. Their absolute solubilities in nonpolar media, including the lipids of the skin barrier, are demonstrably higher, a factor which should favor their percutaneous delivery.

### SPECIFIC EXAMPLE 2

### Percutaneous Delivery of Thalidomide and N-alkyl Analogs

In vitro diffusion cell methods were used to confirm the percutaneous absorption of the compounds and compositions of the present invention.

Chromatography: Amounts of thalidomide and its N-alkyl analogs, which penetrated through skin mounted in diffusion cells were quantitatively determined by HPLC.

Skin Preparation and Permeation: The human cadaver skin used in the permeation studies was obtained from the Anatomical Donation Program at the University of Michigan. Vertical Franz diffusion cells with a 4 ml capacity receptor compartment and a 0.8 cm² diffusion area were used in the permeation studies. The epidermal layer of the skin was mounted carefully onto the lower half of the cells of the difflusion apparatus with the stratum corneum facing up. The receptor compartments were filled with isotonic phosphate buffer (pH 6.4). The temperature of the cell system was maintained at 32° C. by circulating water from a constant temperature water bath through the jacket of the lower compartment of each cell assembly. To begin an experiment, the donor compartment was charged with 300 μl of fresh prepared saturated solution of the drug and covered immediately with Parafilm to prevent any significant evaporation of volatile components of the applied medium during the absorption experiment. At predetermined times, samples were taken and were directly assayed by HPLC to determine the drug concentration of each. Care was taken to maintain skin conditions.

The solubilities of thalidomide and its N-alkyl analogs in the vehicles used for delivery were obtained by equilibrating excess amounts of each of the compounds with each of the media used as vehicles, including buffered water, pure alkanols and solvent mixtures with and without enhancer.

The permeability coefficient for a given run was calculated from Fick's law of diffuision:

$$P = \frac{{V}_{R} ( dC / dt )}{A ( Δ C )}$$

where:

dC/dt is the steady-state slope of a plot of the amount of substance which had penetrated the skin against time in terms of jig/h. It was determined by taking the ratio of the total amount permeated in an interval of time to the length of the time interval.

P is the effective permeability coefficient (cm/h) which is calculated.

A is the difflusional area, which was 0.8 cm².

ΔC is the concentration differential existing across the membrane. This was effectively equal to the saturation concentration in the donor phase (μg/ml) as, through total exchange sampling, a near zero receiver concentration (sink condition) was closely approximated. AC is, in effect, the thermodynamic force driving mass transfer. The maximum driving force is seen at the saturation solubility (excluding supersaturation).

V_{R} is the volume of the receiver compartment (4 ml).

To determine the partition coefficients of the compound, thalidomide and its analogs 20 were equilibrated between equal volumes of n-octanol and phosphate buffer. Partition coefficients (K_{oct}) were calculated as the ratio of drug concentration in the octanol phase to that in the buffer phase.

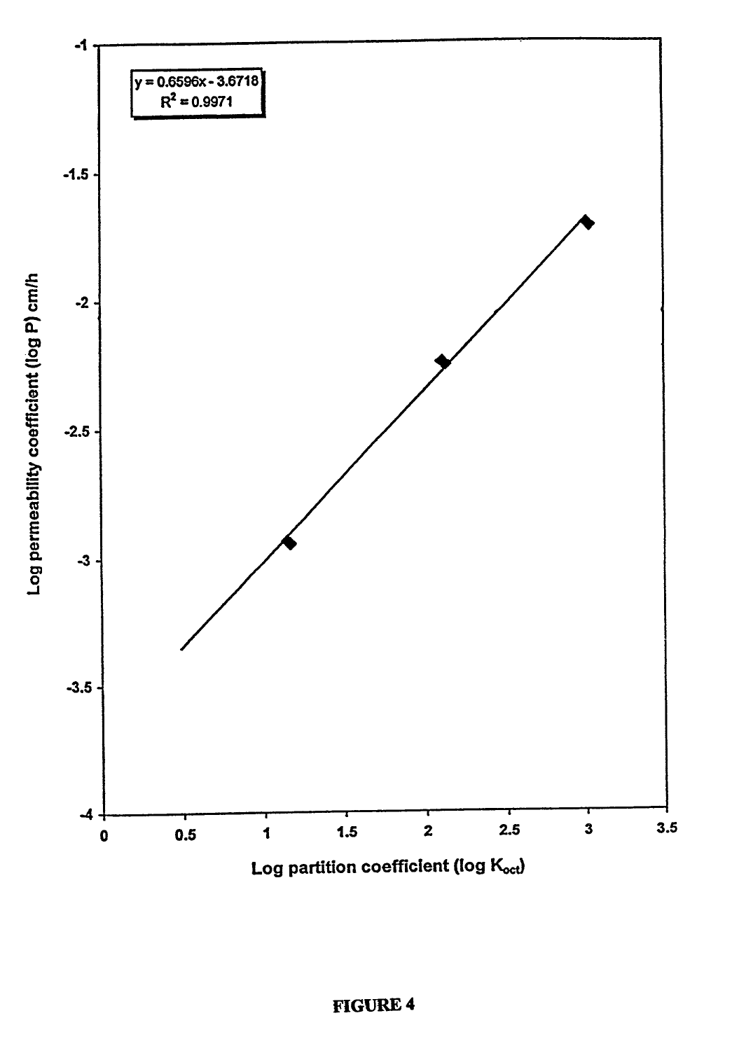

The solubility of a drug in aqueous media used in the donor phase was determined at 32° C. This was done in order to subsequently assess the permeability coefficient using saturated solutions. The solubilities measured at 32° C. were found to be higher but of the same order of magnitude as those determined at 25° C. (see Table 2 for the latter). While the methylene group sensitivities are clearly not the same, when the experimental permeability coefficients from water are plotted against the partition coefficients (FIG. 4) a strong correlation is found between them. This correlation reflects the fact that skin partitioning is an element of the mass transport process. This is consistent with the generally accepted fact that the skin acts as a first good approximation as a lipophilic barrier.

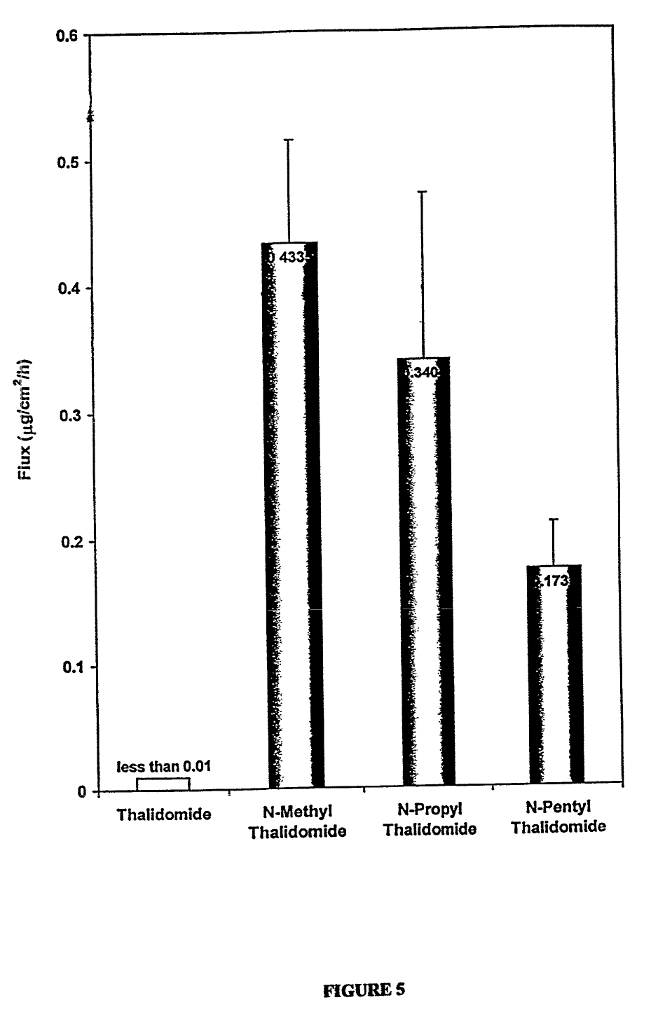

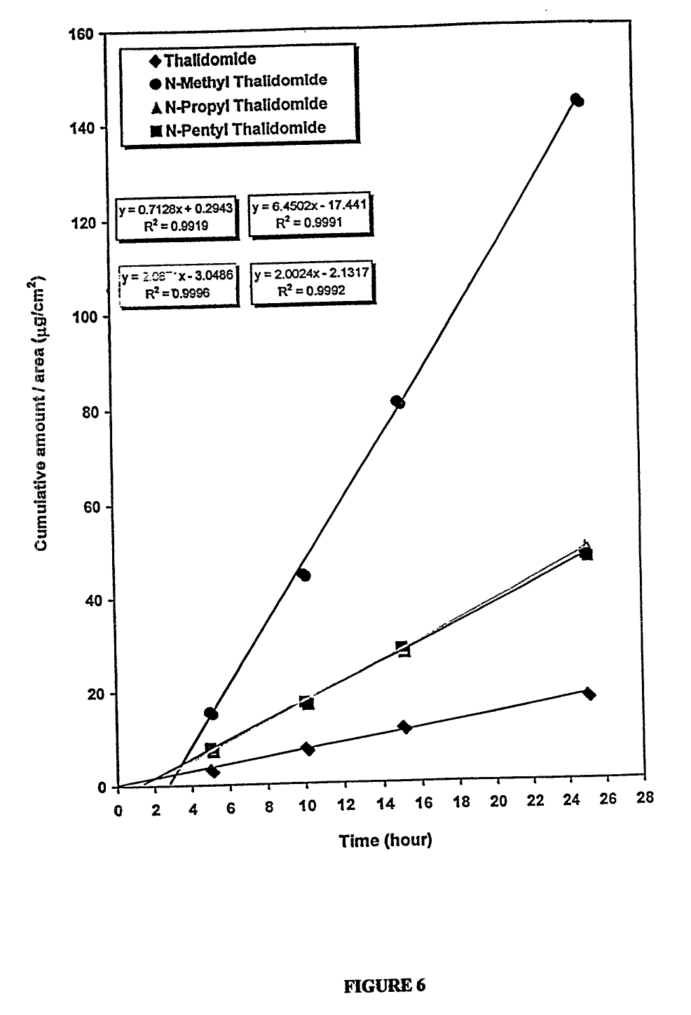

The permeation parameters (flux, J; lag time, T_{L} and permeability coefficient, P) of thalidomide and its N-alkyl analogs from their saturated aqueous solutions (pH 6.0) and an ethanol/water/octanol vehicle are summarized in Table 4. The permeation data were plotted as the cumulative amount of drug penetrated through skin as a function of time. The steady-state flux was determined from the slope of the linear portion of the cumulative amount-time plot. The lag time (T_{L}) was determined by extrapolating the linear portion of the curve to its intersection with the time axis. None of the compounds evidenced detectable lag times within the extended time frames of the aqueous vehicle permeation experiments. A bar plot of the mean steady-state flux and standard deviations (SD) for thalidomide and its N-alkyl analogs from water can be seen in FIG. 5. Since thalidomide was actually not detected, its greatest possible mean steady-state flux from water was calculated according to the limit of detection of the HPLC method, which was 0.01 μg/ml. Thus, the flux of thalidomide was less than 0.01 μg/cm²/h. The fluxes of thalidomide and its N-alkyl analogs were all statistically different from one another (p<0.1). Typical cumulative amount permeated-time profiles for thalidomide and its N-alkyl analogs from the ethanol/water/octanol (Formulation C) vehicle are shown in FIG. 6. In all cases, stable steady-state fluxes were attained within 3 hr after application of the drug solutions.

TABLE 4
Permeation parameters of thalidomide and its N-alkyl analogs through
human skin.

| | | | | P ± SD × 10⁻³ |
|---|---|---|---|---|
| | | J ± SD | T_{L} = SD | (cm/h) |
| Vehicle | Compound | (μg/cm²/h) | (h) | at 32° C. |
| Aqueous | Thalidomide | <0.01 ± 0.00^{b)} | ^{c)} | <0.16 ± 0.00^{b)} |
| (pH 6.0)^{a)} | N-Methyl | 0.43 ± 0.08 | ^{c)} | 1.17 ± 0.22 |
| | N-Propyl | 0.34 ± 0.13 | ^{c)} | 5.73 ± 2.22 |
| | N-Pentyl | 0.18 ± 0.06 | ^{c)} | 19.68 ± 6.31 |
| (EtOH//H₂O/ | Thalidomide | 0.713 ± 0.218 | 2.2 ± 1.4 | |
| octanol)^{d)} | N-Methyl | 6.450 ± 0.448 | 2.8 ± 0.5 | |
| | N-Propyl | 2.087 ± 0.292 | 1.8 ± 1.1 | |
| | N-Pentyl | 2.002 ± 0.178 | 3 ± 1.6 | |

^{a)}Each value from the aqueous donor is the mean ± standard deviation (SD) of 6 diffusion experiments.
^{b)}Since thalidomide could not be detected, these values are calculated according to the limit of detection of the HPLC method (0.01 μg/ml).
^{c)}T_{L} could not be determined accurately because of relatively short lag times.
^{d)}Ethanol/water(pH 6.0)/n-octanol: (57.5:40:2.5). Each value is the mean ± standard deviation (SD) of 3 diffusion experiments.

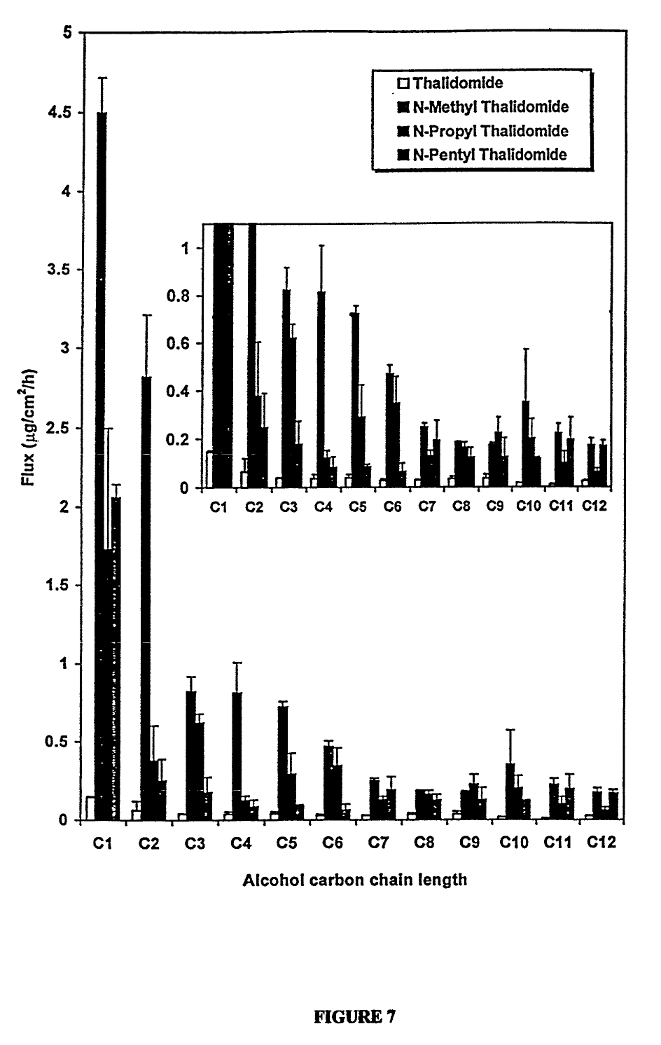

Several compounds are delivered from transdermal systems having alcohol- containing reservoirs. Estradiol and fentanyl are two examples. Consequently, systematic studies were begun using a range of homologous alkanols to explore their possibilities as delivery vehicles. Preparatory to this, the 32° C. solubilities of thalidomide and its N-alkyl analogs in a series of n-alcohols, methanol through dodecanol, were determined (Table 5). The data in Table 5 establish that solubilities across the homologous series of solvents decrease systematically with increasing alkanol chain length. The permeabilities of thalidomide and its N-alkyl analogs through human skin at 32° C. were then determined using the n-alcohols as solvents. These data are presented in FIG. 7 as the steady-state flux (μg/cm²/h) against the number of carbons in the n- alcohols. As was seen from the n-alcohol solubility profiles, the permeabilities of the compounds also decreased, albeit far more irregularly, as the chain length of the alcohol were increased.

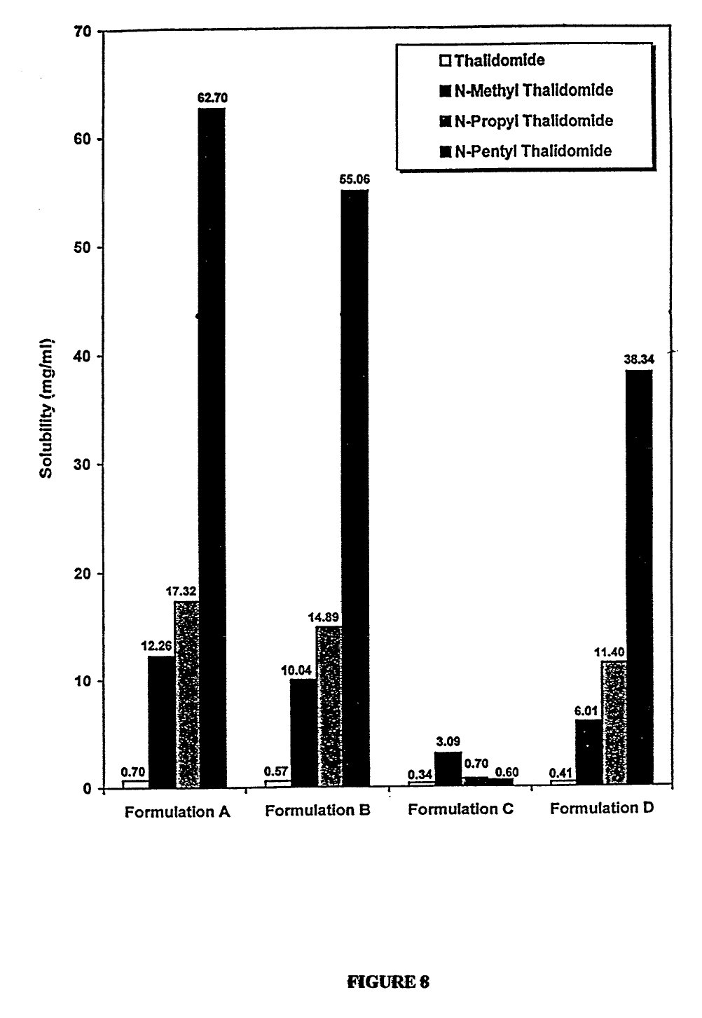

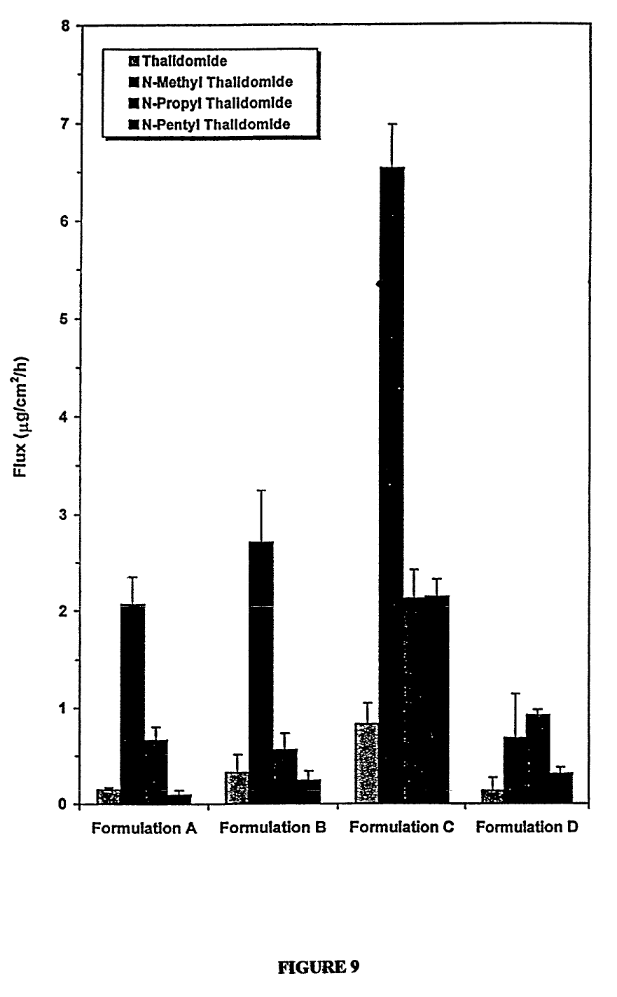

In order to enhance the skin flux and simultaneously determine which analog in the study penetrates the skin best, various solvents and penetration enhancers were combined and used as vehicles. The compositions of these formulations, A-D, are given in Table 6. The formulations were chosen based on the results of experiments aimed at formulating thalidomide into a percutaneous application. The solubilities of thalidomide and its N-alkyl analogs in these formulations are provided in FIG. 8. The steady-state fluxes of thalidomide and its N-alkyl analogs, from formulations A-D can be seen in FIG. 9. The same skin specimen was used for the compounds applied within each individual formulation allowing comparisons of the results for individual compounds. The flux of N-methyl thalidomide is statistically higher (p<0.05) than that of thalidomide and the other analogs in formulations A, B and C. Although the flux of N-methyl thalidomide is not statistically separable (p<0.05) from fluxes obtained for the N-propyl and N-pentyl analogs when using formulation D, all the N-alkyl analogs penetrated the skin more readily than does thalidomide (p<0.05).

TABLE 5
Solubility of thalidomide and its N-alkyl analogs in a series
of n-alcohols.

| | SOLUBILITY (mg/ml) at 32° C. | | | |
|---|---|---|---|---|
| | | N-Methyl | N-Propyl | N-Pentyl |
| n-Alcohol | Thalidomide | Thalidomide | Thalidomide | Thalidomide |
| Methanol | 1.13 | 13.78 | 18.78 | 42.60 |
| Ethanol | 0.40 | 6.91 | 13.20 | 29.63 |
| Propanol | 0.26 | 5.78 | 10.55 | 26.65 |
| Butanol | 0.19 | 4.47 | 10.35 | 26.11 |
| Pentanol | 0.16 | 3.98 | 8.56 | 24.08 |
| Hexanol | 0.12 | 3.26 | 7.26 | 21.78 |
| Heptanol | 0.09 | 2.93 | 6.45 | 20.34 |
| Octanol | 0.07 | 2.64 | 6.21 | 20.19 |
| Nonanol | 0.06 | 2.41 | 5.83 | 15.87 |
| Decanol | 0.05 | 2.38 | 3.87 | 12.53 |
| Undecanol | 0.04 | 2.29 | 3.73 | 11.67 |
| Dodecanol | 0.04 | 1.91 | 3.18 | 11.56 |

TABLE 6
Composition and ratios of formulations A-D.

| Formulation | Solvent/Enhancer | % Composition |
|---|---|---|
| A | Isopropanol | 70 |
| | NMP^{a)} | 10 |
| | n-Octanol | 10 |
| | Citric Acid | 5 |
| | IPM^{b)} | 5 |
| B | Ethanol | 80 |
| | n-Octanol | 10 |
| | Citric Acid | 5 |
| | IPM^{b)} | 5 |
| C | Water^{c)} | 575 |
| | Ethanol | 40 |
| | n-Octanol | 2.5 |
| D | Ethanol | 95 |
| | IPM^{b)} | 5 |

^{a)}N-Methyl Pyrrolidone.
^{b)}Isopropyl Myristate Ester.
^{c)}pH 6.0

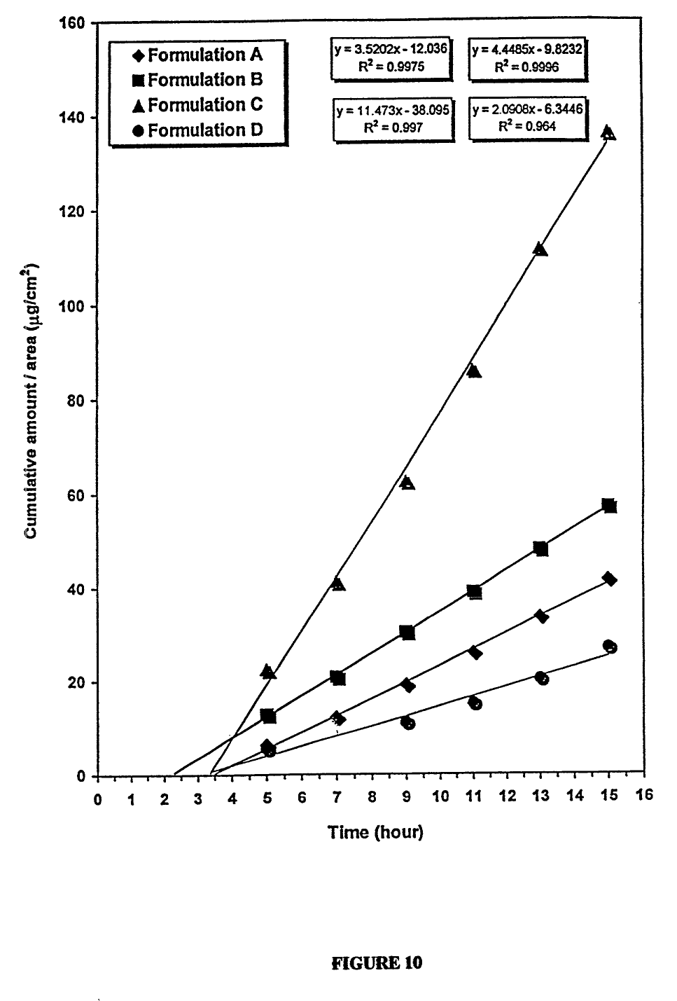

In FIG. 10, N-methyl thalidomide's penetration curves (cumulative amount penetrated versus time) obtained using the four different vehicle compositions (formulations A-D) are shown. There is a distinct lag time and eventually the penetration rate becomes constant. Since all the vehicle compositions were studied using membranes cut from the same skin specimen, results are directly comparable. The maximum steady-state flux obtained for N-methyl thalidomide (11.47 μg/cm²/h) occurred with formulation C. Formulation C proved to be statistically superior (p<0.01) as a delivery medium to formulations A, B and D.

The TNFα inhibitory effects of the N-alkyl analogs of the present invention were investigated by stimulating peripheral blood mononuclear cells in vitro with lipopolysaccharide (LPS). It is known that thalidomide inhibits LPS-induced TNFα production and thus this compound was used as a control. A 50 μl aliquot of each compound in solvent at 50μg/ml was added to a well containing 100-μl of the cell suspension medium. Percentage TNFα inhibition was calculated.

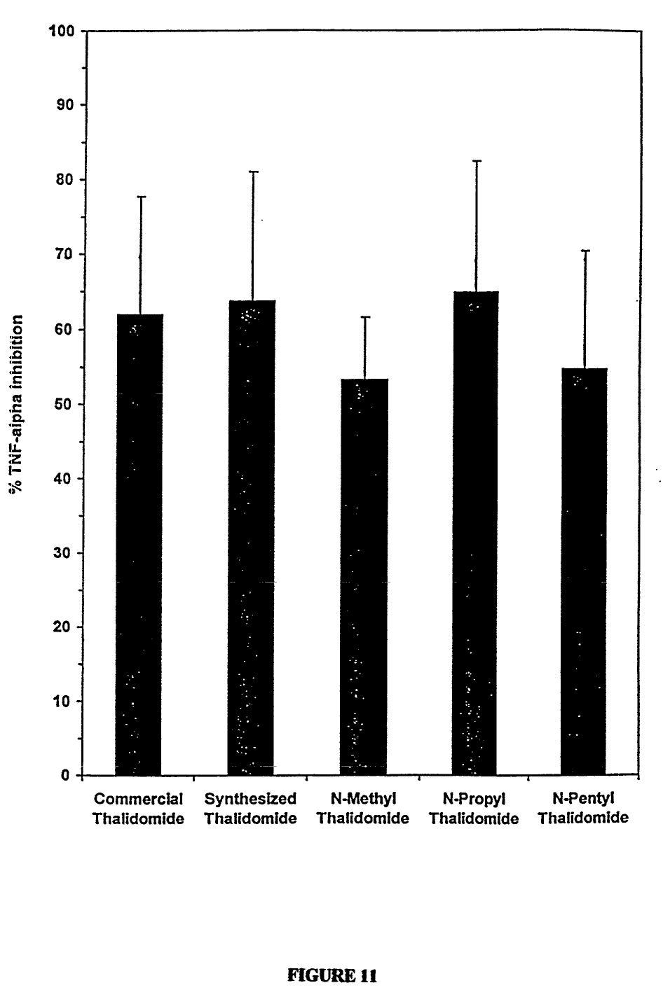

The TNFα inhibitory effects of thalidomide and its N-alkyl analogs were measured in the supernatant of human peripheral blood mononuclear cells (PBMCs) stimulated with LPS. Cultures containing 10⁶ human mononuclear cells were incubated with thalidomide or one of the N-alkyl analogs for 1 hr and then stimulated with 2 μg/ml of LPS for 16 hr. The data on the TNFα effects of thalidomide and its N-alkyl analogs are summarized in FIG. 11. Thalidomide has been shown in previous studies to partially inhibit TNFα production by PBMCs stimulated in vitro with LPS. Sampio, E. P. et al., Journal Of Experimental Medicine 173:699-703 (1991). The addition of N-alkyl groups to the glutarimide ring of the thalidomide molecule:

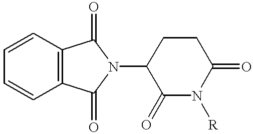

| | | |
|---|---|---|
| R = H | → | Thalidomide |
| R = CH₃ | → | N-Methyl Thalidomide |
| R = CH₂CH₂CH₃ | → | N-Propyl Thalidomide |
| R = CH₂CH₂CH₂CH₂CH₃ | → | N-Pentyl Thalidomide |

did not change the compounds ability to inhibit TNFα production, i.e, inhibition was at a level similar to that observed with thalidomide (see FIG. 11). The activities of the compounds are not significantly different.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

Patent and literature references cited herein are incorporated by reference as if fully set forth.

We claim:

1. A method of treating a mammal having an inflammatory disease, comprising the step of topically administering to the mammal a compound selected from the group consisting of thalidomide, an analog of thalidomide, and combinations therefore.

2. The method of claim 1, wherein the compound is thalidomide.

3. The method of claim 1, wherein the compound is an analog of thalidomide.

4. The method of claim 3, wherein the analog of thalidomide is an N-alkyl analog of thalidomide.

5. The method of claim 4, wherein the N-alkyl analog is C₁-C₁₀.

6. The method of claim 1, wherein the inflammatory disease is inflammatory arthritis.

7. The method of claim 1, wherein the inflammatory disease is cutaneous manifestations of systemic lupus erythematosus.

8. The method of claim 1, wherein the inflammatory disease is psoriasis.

9. The method of claim 1, wherein the inflammatory disease in Bechet's disease.

10. The method of claim 6, wherein the inflammatory arthritis is rheumatoid arthritis.

11. The method of claim 1, wherein the mammal is a human.

12. A composition comprising a dermatologically acceptable carrier for percutaneous delivery and a compound selected from the group consisting of thalidomide, an analog of thalidomide, and combinations thereof.

13. The composition of claim 12, wherein the compound is thalidomide.

14. The composition of claim 12, wherein the compound is an analog of thalidomide.

15. The composition of claim 14, wherein the analog of thalidomide is an N-alkyl analog of thalidomide.

16. The composition of claim 15, wherein the N-alkyl analog of thalidomide is C₁-C₁₀.

17. The composition of claim 12, further comprising a percutaneous penetration enhancer.

18. The composition of claim 12, wherein the percutaneous penetration enhancer is a chemical enhancer.

19. The composition of claim 12, wherein the percutaneous penetration enhancer is a physical enhancer.
